(19) United States
(12) Patent Application Publication
(10) Pub. No.: US 2019/0038724 A1
(43) Pub. Date: Feb. 7, 2019

(54) Adeno-Associated Virus-Mediated CRISPR-Cas9 Treatment of Ocular Disease

(71) Applicant: SPARK THERAPEUTICS, INC., Philadelphia, PA (US)

(72) Inventors: George Buchlis, Philadelphia, PA (US); Xavier Anguela, Philadelphia, PA (US); Katherine A. High, Merion Station, PA (US)

(73) Assignee: SPARK THERAPEUTICS, INC., Philadelphia, PA (US)

(21) Appl. No.: 16/156,649

(22) Filed: Oct. 10, 2018

Related U.S. Application Data
(63) Continuation of application No. 15/143,272, filed on Apr. 29, 2016, now abandoned.
(60) Provisional application No. 62/156,025, filed on May 1, 2015.

(51) Int. Cl. A61K 38/46; C12N 15/10; C12N 9/22; A61K 31/7088

(52) U.S. Cl. CPC A61K 38/465; C12N 15/102; C12N 9/22; A61K 48/00; C12N 2750/14143; A61K 31/7088

(57) ABSTRACT

Disclosed herein are compositions and methods of treating and/or correcting ocular disease in a subject, such as a mammal (e.g., human) eye using an Adeno-associated virus (AAV) system. The AAV system employs a nucleic acid encoding a CRISPR-Cas9 system for targeted gene disruption or correction.

### RELATED APPLICATION INFORMATION

This application is a continuation application of U.S. application Ser. No. 15/143,272, filed Apr. 29, 2016, which claims the benefit of priority to U.S. Provisional Application Ser. No. 62/156,025, filed May 1, 2015, all of which applications are expressly incorporated herein by reference in their entirety.

### INTRODUCTION

Successful gene transfer for monogenic human disease can potentially provide a singularly administered, lifelong cure. Gene transfer, also termed gene therapy, arises from the idea that a monogenic disease can be corrected by the exogenous introduction of the missing or otherwise compromised genetic material. There are generally two ways to achieve this goal. In gene addition, a vector encoding the gene of interest is delivered and expressed in the host, without altering the endogenous, mutated gene locus. This is the most common gene therapy approach currently under investigation, and requires that the genetic material carry an appropriate promoter to drive its transcription. In gene correction, a specific double-stranded break (DSB) is induced at the mutated locus, allowing homologous regions flanking the transgene cassette to recombine with the host locus and replace the mutated DNA with the correct sequence at the site of the DSB. The advantage to this strategy is the correction of the endogenous locus, which is under the physiological control of its own promoter and can more appropriately dictate the rate and timing of transcription.

Adeno-associated virus was first discovered in the mid 1960's as a contaminant of viral preparations of adenovirus[1]. Since then, progressively safer and more effective methods to use AAV as a recombinant DNA vector have been developed[2-5]. AAV has a single stranded, 4.7 kb genome encoding replication (rep) and capsid (cap) genes. It is predominantly non-integrating, and forms stable episomes in non-dividing tissue. In spite of its high seroprevalence in the adult human population (80% for AAV2), it has not been associated with any human disease[6]. Thus its stable expression in tissues such as muscle, eye, and liver, its lack of pathogenicity, and its ease of high titer production have made it a very attractive and popular gene transfer platform.

Treatment of genetic diseases of the eye, e.g., inherited genetic diseases of the eye, such as diseases that cause blindness, remains a problem. Examples of such are retinitis pigmentosa, maculopathies, Leber's congenital amaurosis, early onset severe retinal dystrophy, achromatopsia, retinoschisis, ocular albinism, oculocutaneous albinism, glaucoma, Stargardt disease, choroideremia, age-related macular degeneration, Spinocerebellar Ataxia Type 7(SCA 7), color blindness, and lysosomal storage diseases that affect the cornea, such as Mucopolysaccharidosis (MPS) IV and MPS VII. Thus, therapies for genetic diseases of the eye need to be developed.

AAV-mediated delivery to the human retina reverses blindness in patients with mutations in retinal pigment epithelium 65 (RPE65), in Leber's Congenital Amaurosis. Efficacy and safety with this product[7-9] was shown over a range of years and ages.

### SUMMARY

Disclosed herein are compositions and methods of correcting ocular disease in a subject, such as a mammal (e.g., human) eye using an Adeno-associated virus (AAV) system. The AAV system employs a nucleic acid encoding a CRISPR-Cas9 system[10-15] for targeted gene disruption or correction.

In one embodiment, this system will employ 3 AAV vectors: one encoding Cas9 or a functional ortholog, one containing the guide RNA sequence for targeted cleavage, and one containing the donor cDNA sequence of the mutated gene to be inserted at the cleavage site. The donor to Cas9 construct administration ratios can range anywhere from 1:1 to 5:1.

In another embodiment, this system will employ 2 AAV vectors: one encoding a Cas9 ortholog less than 3.5 kb in length and will have the guide RNA encoded in cis, and one vector containing the donor cDNA sequence of the mutated gene to be inserted at the cleavage site. For genes greater than 4.8 kb, this donor will contain either the 3′ cDNA portion of the gene up to 4.8 kb allowing correction upstream of the majority of the mutated gene (FIG. 1), or the 5′ promoter and upstream cDNA portion of the gene, which will then splice to the correct downstream sequence (FIG. 2).

In a further embodiment, this system will employ 2 AAV vectors: one encoding a one encoding Cas9 or a functional ortholog, and one containing a guide RNA sequence specific for cleavage of a target gene.

In yet another embodiment, this system will employ one AAV vector, the vector comprising a nucleic acid encoding a functional Type II CRISPR-Cas9 and a guide RNA specific for cleavage of a target gene.

### DESCRIPTION OF DRAWINGS

FIG. 1: The endogenous locus of an oversized, mutated target gene is cleaved upstream of the known mutation by a guided Cas9 nuclease delivered by AAV. A donor AAV construct containing an inverted terminal repeat (ITR), a splice acceptor signal, correct cDNA sequence, polyadenylation signal, and ITR, is inserted into the locus at the cut site. After transcription and mRNA processing, the correct mRNA template will be available for protein translation.

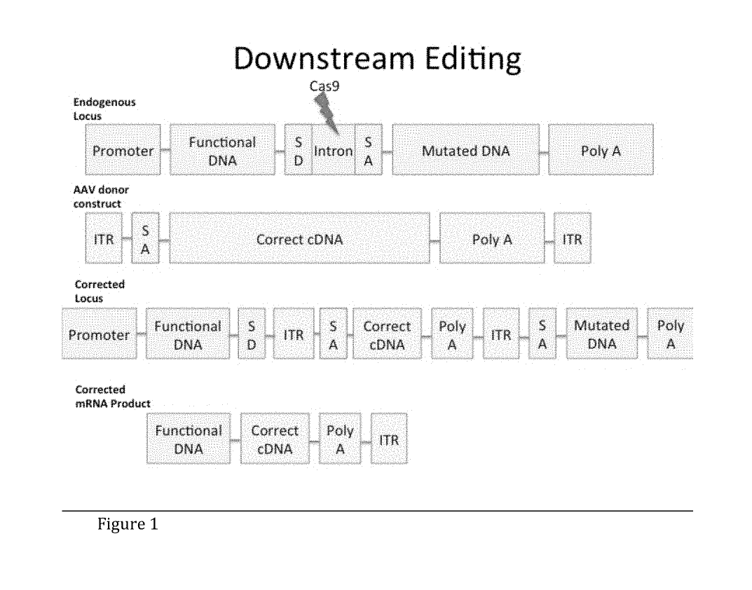

FIG. 2: The endogenous locus of an oversized, mutated target gene is cleaved downstream of the known mutation by a guided Cas9 nuclease delivered by AAV. A donor AAV construct containing an inverted terminal repeat (ITR), a splice acceptor signal, a polyadenylation signal, a ubiquitous promoter, the correct cDNA sequence, splice donor signal, and ITR, is inserted into the locus at the cut site. After transcription and mRNA processing, the correct mRNA template will be available for protein translation.

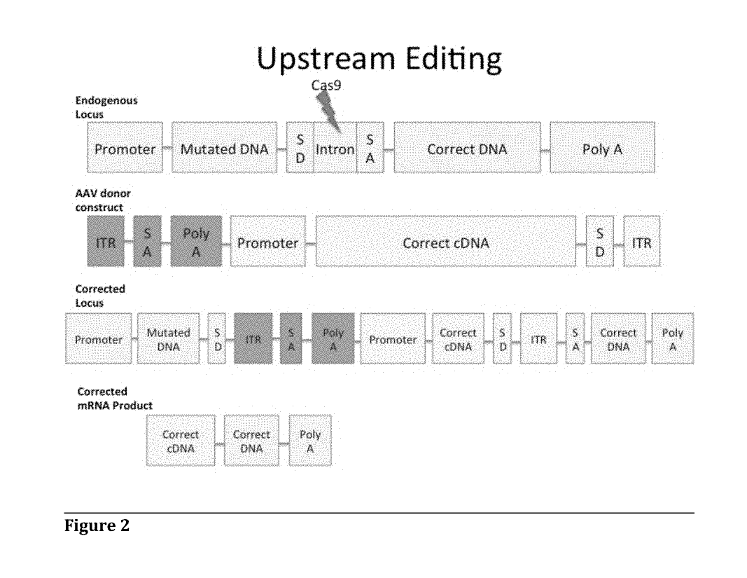

### DETAILED DESCRIPTION

The Clustered Regularly Interspaced Short Palindromic Repeat/Cas (CRISPR/Cas) system, by way of the Cas9 nucleases, can be directed by short RNAs to induce precise cleavage at endogenous target genes in genomic DNA, and can edit multiple sites on the genome by allowing for coding of several sequences in a single CRISPR array. A single Cas enzyme can be programmed by a short RNA molecule (referred to as the “guide” RNA) to recognize a target DNA. In other words, the Cas enzyme can be recruited to a specific target DNA using said short RNA molecule- the guide RNA- to provide for specificity of the CRISPR-mediated nucleic acid cleavage.

There are three CRISPR types, the most commonly used type for gene correction or disruption to date is type II. For example, the CRISPR RNA targeting sequences are transcribed from DNA sequences clustered within the CRISPR array. In order to operate, the CRISPR targeting RNA, or precursor crRNA (pre-crRNA), is transcribed and the RNA is processed to separate the individual RNAs (crRNAs) dependent on the presence of a trans-activating CRISPR RNA (tracrRNA) that has sequence complementarity to the CRISPR repeat. When the trans RNA hybridizes to the CRISPR repeat, it initiates processing by the double-stranded RNA specific ribonuclease, RNAse III, forming tandem tracrRNA:crRNA duplexes, which can be synthetically made as single guide RNAs (sgRNAs) for genome engineering purposes. The Cas9 nuclease, which is activated and responds specifically to the DNA sequence complementary to the crRNA and cleaves it. A target sequence must contain a specific sequence on its 3′ end, called the protospacer adjacent motif (PAM) in the DNA to be cleaved which is not present in the CRISPR RNA that recognizes the target sequence.

In addition to the naturally occurring guide RNAs, synthetic guide RNAs can be fused to a CRISPR vector. The design of guide RNAs with target-recognition sequences and other essential elements (e.g., hairpin and scaffold sequence) using bioinformatics methods is described (see, e.g., Mali et al., Science 339: 823-826 (2013)).

The invention provides compositions and methods of disrupting, correcting or replacing a target gene in a eukaryotic cell. In some embodiments, a composition includes a plurality of AAV vectors comprising various elements of a CRISPR system. In one representative embodiment, a first AAV vector includes a nucleic acid encoding a functional Type II CRISPR-Cas9 (enzyme); and a second AAV vector includes a guide RNA sequence for a target gene to allow disruption of the target gene. In another representative embodiment, a first AAV vector includes a nucleic acid encoding a functional Type II CRISPR-Cas9 (enzyme); a second AAV vector includes a guide RNA sequence for a target gene; and a third AAV vector includes a donor nucleic acid sequence for correction or replacement of a target gene. In another representative embodiment, a first AAV vector includes a nucleic acid encoding a functional Type II CRISPR-Cas9 (enzyme) and a guide RNA sequence for a target gene; and a second AAV vector includes a donor nucleic acid sequence for correction or replacement of a target gene. In a further representative embodiment, a single AAV vector includes a nucleic acid encoding a functional Type II CRISPR-Cas9 (enzyme) and a guide RNA sequence for a target gene to allow disruption of the target gene.

In a representative method, the method comprises providing one or more AAV vectors (typically 2 or 3 AAV vectors) comprising elements of a CRISPR system, to bind to the target gene to effect cleavage of said target polynucleotide thereby modifying the target gene such as disrupting the target gene or correcting or replacing all or a part of the target gene with a donor nucleic acid. Elements of said CRISPR system include a CRISPR enzyme, which can be complexed with a guide RNA sequence, said guide RNA which can be hybridized to a target sequence within said target gene. Cleavage at the target gene can involve cleaving one or two strands by the CRISPR enzyme. In some embodiments, a method includes correcting or replacing the cleaved target gene by introduction of a donor nucleic acid, which donor nucleic acid encodes a protein that corrects for the mutated or defective target gene.

A Cas gene as described herein includes, but is not limited to, Cas3 or Cas9. The enzyme may be a Cas9 homolog or ortholog. Cas9 orthologs include Cas9 from Streptococcus pyogenes, Neisseria meningitidis, Streptococcus thermophilus, Streptococcus pneumnoniae, Campylobacter coli, Campylobacter jejuni, Streptococcus mutans, Pasteurella multocida, Bifidobacterium longum, Bacillus smithii, Treponema denticola, mycoplasma canis and enterococcus faecalis. A Cas9 may include mutated Cas9 derived from these organisms.

Exemplary AAV vectors include capsid sequence of any of AAV1, AAV2, AAV3, AAV4, AAV5, AAV6, AAV7, AAV8, AAV9, AAV10, AAV11, Rh10, Rh74 or AAV-2i8, or a capsid variant of AAV1, AAV2, AAV3, AAV4, AAV5, AAV6, AAV7, AAV8, AAV9, AAV10, AAV11, Rh10, Rh74 or AAV-2i8. Recombinant AAV vectors of the invention also include AAV1, AAV2, AAV3, AAV4, AAV5, AAV6, AAV7, AAV8, AAV9, AAV10, AAV11, Rh10, Rh74 or AAV-2i8, and variants thereof. Particular capsid variants include capsid variants of AAV1, AAV2, AAV3, AAV4, AAV5, AAV6, AAV7, AAV8, AAV9, AAV10, AAV11, Rh10, Rh74 or AAV-2i8, such as a capsid sequence with an amino acid substitution, deletion or insertion/addition.

AAV vectors can include additional elements that function in cis or in trans. In particular embodiments, an AAV vector that includes a vector genome also has: one or more inverted terminal repeat (ITR) sequences that flank the 5′ or 3′ terminus of the donor sequence; an expression control element that drives transcription (e.g., a promoter or enhancer) of the donor sequence, such as a constitutive or regulatable control element, or tissue-specific expression control element; an intron sequence, a stuffer or filler polynucleotide sequence; and/or a poly-Adenine sequence located 3′ of the donor sequence.

Typically, expression control elements are nucleic acid sequence(s) that influence expression of an operably linked polynucleotide. Control elements, including expression control elements as set forth herein such as promoters and enhancers, present within a vector are included to facilitate proper nucleic acid transcription and translation (e.g., a promoter, enhancer, splicing signal for introns, maintenance of the correct reading frame of the gene to permit in-frame translation of mRNA and, stop codons etc.), and AAV packaging. Such elements typically act in cis, referred to as a “cis acting” element, but may also act in trans.

Expression control can be effected at the level of transcription, translation, splicing, message stability, etc. Typically, an expression control element that modulates transcription is juxtaposed near the 5′ end (i.e., “upstream”) of a transcribed nucleic acid. Expression control elements can also be located at the 3′ end (i.e., “downstream”) of the transcribed sequence or within the transcript (e.g., in an intron). Expression control elements can be located adjacent to or at a distance away from the transcribed sequence. Typically, owing to the polynucleotide length limitations of certain vectors, such as AAV vectors, such expression control elements will be within 1 to 1000 nucleotides from the transcribed nucleic acid.

A “promoter” as used herein can refer to a nucleic acid (e.g., DNA) sequence that is located adjacent to a polynucloetide sequence that encodes a recombinant product. A promoter is typically operatively linked to an adjacent sequence, and increases an amount expressed from a nucleic acid as compared to an amount expressed when no promoter exists.

An “enhancer” as used herein can refer to a sequence that is located adjacent to the nucleic acid. Enhancer elements are typically located upstream of a promoter element but also function and can be located downstream of or within a DNA sequence (e.g., a donor nucleic acid). Hence, an enhancer element can be located 100 base pairs, 200 base pairs, or 300 or more base pairs upstream or downstream of a nucleic acid. Enhancer elements also typically increase expression of a nucleic acid.

Expression control elements include ubiquitous or promiscuous promoters/enhancers which are capable of driving expression of a polynucleotide in many different cell types. Such elements include, but are not limited to the cytomegalovirus (CMV) immediate early promoter/enhancer sequences, the Rous sarcoma virus (RSV) promoter/enhancer sequences and the other viral promoters/enhancers active in a variety of mammalian cell types, or synthetic elements that are not present in nature (see, e.g., Boshart et al, Cell, 41:521-530 (1985)), the SV40 promoter, the dihydrofolate reductase (DHFR) promoter, the cytoplasmic β-actin promoter and the phosphoglycerol kinase (PGK) promoter.

Expression control elements include those active in a particular tissue or cell type, referred to herein as a “tissue-specific expression control elements/promoters.” Tissue-specific expression control elements are typically active in specific cell or tissue (e.g., eye, retina, central nervous system, spinal cord, eye, retina, etc.). Expression control elements are typically active in these cells, tissues or organs because they are recognized by transcriptional activator proteins, or other regulators of transcription, that are unique to a specific cell, tissue or organ type.

Expression control elements also can confer expression in a manner that is regulatable, that is, a signal or stimuli increases or decreases expression of the operably linked nucleic acid. A regulatable element that increases expression of the operably linked nucleic acid in response to a signal or stimuli is also referred to as an “inducible element” (i.e., is induced by a signal). Particular examples include, but are not limited to, a hormone (e.g., steroid) inducible promoter. A regulatable element that decreases expression of the operably linked nucleic acid in response to a signal or stimuli is referred to as a “repressible element” (i.e., the signal decreases expression such that when the signal, is removed or absent, expression is increased). Typically, the amount of increase or decrease conferred by such elements is proportional to the amount of signal or stimuli present; the greater the amount of signal or stimuli, the greater the increase or decrease in expression.

Expression control elements also include the native elements(s). A native control element (e.g., promoter) may be used when it is desired that expression of the nucleic acid may mimic the native expression. A native element may be used when expression of the nucleic acid is to be regulated temporally or developmentally, or in a tissue-specific manner, or in response to specific transcriptional stimuli. Other native expression control elements, such as introns, polyadenylation sites or Kozak consensus sequences may also be used.

As used herein, the term “operable linkage” or “operably linked” refers to a physical or functional juxtaposition of the components so described as to permit them to function in their intended manner. In the example of an expression control element in operable linkage with a nucleic acid, the relationship is such that the control element modulates expression of the nucleic acid. More specifically, for example, two DNA sequences operably linked means that the two DNAs are arranged (cis or trans) in such a relationship that at least one of the DNA sequences is able to exert a physiological effect upon the other sequence.

AAV vectors may include filler or stuffer polynucleotide sequence. For example, where a donor nucleic acid has a length less than about 4.7 Kb. A filler or stuffer polynucleotide sequence has a length that when combined with donor nucleic acid the total combined length is between about 3.0-5.5 Kb, or between about 4.0-5.0 Kb, or between about 4.3-4.8 Kb.

Filler or stuffer polynucleotide sequences can be located in the vector sequence at any desired position such that it does not prevent a function or activity of the vector. In one aspect, a filler or stuffer polynucleotide sequence is positioned between a 5′ and/or 3′ ITR (e.g., an ITR of AAV1, AAV2, AAV3, AAV4, AAV5, AAV6, AAV7, AAV8, AAV9, AAV10, AAV11, Rh10, Rh74 or AAV-2i8, and variants thereof) that flanks the respective 5′ and/or 3′ termini of a donor nucleic acid sequence.

Typically, a filler or stuffer polynucleotide sequence is inert or innocuous and has no function or activity. In various particular aspects, a filler or stuffer polynucleotide sequence is not a bacterial polynucleotide sequence, a filler or stuffer polynucleotide sequence is not a sequence that encodes a protein or peptide, a filler or stuffer polynucleotide sequence is a sequence distinct from any of: the donor sequence, an AAV inverted terminal repeat (ITR) sequence, an expression control element, or a poly-adenylation (poly-A) signal sequence. In various particular aspects, a filler or stuffer polynucleotide sequence is an intron sequence that is related to or unrelated to the donor sequence.

An intron can also function as a filler or stuffer polynucleotide sequence in order to achieve a length for AAV vector packaging into a virus particle. Introns and intron fragments (e.g. portion of intron I of FIX) that function as a filler or stuffer polynucleotide sequence also can enhance expression. Inclusion of an intron element may enhance expression compared with expression in the absence of the intron element (Kurachi et al., 1995, supra).

The use of introns is not limited to naturally occurring genomic sequence, and can include introns associated with a completely different gene or other DNA sequence. Accordingly, other untranslated (non-protein encoding) regions of nucleic acid, such as introns found in genomic sequences from cognate (related) genes and non-cognate (unrelated) genes can also function as filler or stuffer polynucleotide sequences in accordance with the invention.

The term “nucleic acid” is used herein to refer to all forms of nucleic acid, polynucleotides and oligonucleotides, including deoxyribonucleic acid (DNA) and ribonucleic acid (RNA). Nucleic acids include genomic DNA, cDNA and RNA. Polynucleotides include naturally occurring, synthetic, and intentionally modified or altered polynucleotides. Polynucleotides can be single, double, or triplex, linear or circular, and can be of any length. A sequence or structure of a particular polynucleotide may be described herein according to the convention of providing the sequence in the 5′ to 3′ direction.

A “donor” nucleic acid refers to a polynucleotide inserted into an AAV vector for purposes of vector mediated transfer/delivery of the polynucleotide into a cell. Once transferred/delivered into the cell, the polynucleotide within the vector, can be expressed (e.g., transcribed, and translated if appropriate). An example of a donor nucleic acid sequence would be a gene, or cDNA as set forth in Table 1.

The “polypeptides,” “proteins” and “peptides” encoded by the “nucleic acids,” including donor nucleic acids, full-length native sequences, as with naturally occurring proteins, as well as functional subsequences, modified forms or sequence variants so long as the subsequence, modified form or variant retains some degree of functionality of the native (wild-type) full-length protein. In the compositions, methods and uses of the invention, such polypeptides, proteins and peptides encoded by nucleic acids, including donor nucleic acids, can be but are not required to be identical to the endogenous (target) gene that is mutated or defective, or encodes a protein having defective or partial function or activity, or whose expression is insufficient, or deficient in the treated mammal. Accordingly, donor nucleic acids in accordance with the invention encode full-length native proteins, as well as partial or functional subsequences, modified forms or sequence variants so long as the subsequence, modified form or variant retains some degree of functionality.

Donor nucleic acids, expression control elements, ITRs, poly A sequences, filler or stuffer polynucleotide sequences can vary in length. In particular aspects, a sequence between about 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000 or more nucleotides in length up to the limit of AAV packaging size limit.

A “transgene” can be used herein to conveniently refer to a donor nucleic acid that is intended or has been introduced into a cell or organism. Transgenes include any gene, such as a gene or cDNA set forth in Table 1.

In a cell having a transgene, the transgene has been introduced/transferred by way of AAV vector. The terms “transduce” and “transfect” refers to introduction of a molecule such as a nucleic acid into a cell or host organism. Accordingly, a transduced cell (e.g., in a mammal, such as a cell or tissue or organ cell), means a genetic change in a cell following incorporation of an exogenous molecule, for example, a polynucleotide or protein (e.g., a transgene) into the cell. Thus, a “transduced” cell is a cell into which, or a progeny thereof in which an exogenous molecule has been introduced. The cell(s) containing the introduced donor nucleic acid (e.g., transgene) can express protein. In methods and uses of the invention, a transduced cell can be in a subject.

Methods and uses of the invention provide a means for delivering (transducing) donor nucleic acid (transgenes) into host cells, including dividing and/or non-dividing cells. The AAV vectors, methods, uses and pharmaceutical formulations of the invention are additionally useful in a method of delivering, administering or providing a nucleic acid, or protein to a subject in need thereof, as a method of treatment. In this manner, the nucleic acid is transcribed and the protein may be produced in vivo in a subject. The subject may benefit from or be in need of the nucleic acid or protein because the subject has a deficiency of the nucleic acid or protein, or because production of the nucleic acid or protein in the subject may impart some therapeutic effect, as a method of treatment or otherwise.

In various embodiments, the AAV vectors are delivered to the eukaryotic cell in a subject. Subjects are typically animals and include human and veterinary applications. Suitable subjects therefore include mammals, such as humans, as well as non-human mammals (e.g., primates). Other subjects include primates (apes, gibbons, gorillas, chimpanzees, orangutans, macaques), a domestic animal (dogs and cats), a farm animal (poultry such as chickens and ducks, horses, cows, goats, sheep, pigs), and experimental animals (mouse, rat, rabbit, guinea pig). Human subjects include fetal, neonatal, infant, juvenile and adult subjects. Subjects include animal disease models, for example, mouse and other animal models of blood clotting diseases and others known to those of skill in the art.

Subjects appropriate for treatment include those having or at risk of producing an insufficient amount or having a deficiency in a functional gene product (protein), or produce an aberrant, partially functional or non-functional gene product (protein), which can lead to disease. In particular embodiments, a subject that would benefit from or is in need of disrupting, correcting or replacing a defective gene, or is in need of disrupting, correcting or replacing a gene encoding a protein having defective or partial function or activity. More particular examples of subjects include those having an ocular disease or disorder caused by a lack of expression or function, or insufficient activity, of one or more proteins. Non-limiting examples of such diseases are set forth in Table 1. Accordingly, subjects include those afflicted or at risk of developing ocular and other diseases set forth in Table 1.

In a particular embodiment, a subject is a human infant. In another particular embodiment, a subject is a human newborn. In a further particular embodiment, a subject is a human between the ages of 1 and 5 years old.

Cells that may be transduced include a cell of any tissue or organ type, of any origin (e.g., mesoderm, ectoderm or endoderm). Non-limiting examples of cells include ocular cells (retinal, corneal, scleral or choroid), or central or peripheral nervous system, such as brain (e.g., neural, glial or ependymal cells). Additional examples include stem cells, such as pluripotent or multipotent progenitor cells that develop or differentiate into any of the foregoing cells.

Invention AAV vectors, methods and uses permit the treatment of genetic diseases. In general, disease states fall into two classes: deficiency states, usually of enzymes, which are generally inherited in a recessive manner, and unbalanced states, at least sometimes involving regulatory or structural proteins, which are inherited in a dominant manner. For deficiency state diseases, transfer of donor nucleic acid to a subject could provide a normal gene into affected tissues for replacement therapy. For unbalanced disease states, transfer of donor nucleic acid could be used to provide a functional protein which could restore or at least amerliorate the unbalanced state.

Methods and uses of the invention include treatment methods, which result in any therapeutic or beneficial effect. In particular aspects of invention methods and uses disclosed herein, expression of the nucleic acid provides a therapeutic benefit to the mammal (e.g., human). In various invention methods and uses, further included are inhibiting, decreasing or reducing one or more adverse (e.g., physical) symptoms, disorders, illnesses, diseases or complications caused by or associated with the disease, or reduced dosage of a supplemental protein.

A therapeutic or beneficial effect of treatment is therefore any objective or subjective measurable or detectable improvement or benefit provided to a particular subject. A therapeutic or beneficial effect can but need not be complete ablation of all or any particular adverse symptom, disorder, illness, or complication of a disease. Thus, a satisfactory clinical endpoint is achieved when there is an incremental improvement or a partial reduction in an adverse symptom, disorder, illness, or complication caused by or associated with a disease, or an inhibition, decrease, reduction, suppression, prevention, limit or control of worsening or progression of one or more adverse symptoms, disorders, illnesses, or complications caused by or associated with the disease, over a short or long duration (hours, days, weeks, months, etc.).

The dose to achieve a therapeutic effect, e.g., the dose in vector genomes/per kilogram of body weight (vg/kg), will vary based on several factors including, but not limited to: route of administration, the nucleic acid expression required to achieve a therapeutic effect, the specific disease treated, any host immune response to the vector, and the stability of the protein expressed. One skilled in the art can determine a rAAV/vector genome dose range to treat a patient having a particular disease or disorder based on the aforementioned factors, as well as other factors.

Administration or in vivo delivery to a subject can be performed prior to development of an adverse symptom, condition, complication, etc. caused by or associated with the disease. For example, a screen (e.g., genetic) can be used to identify such subjects as candidates for invention compositions, methods and uses. Such subjects therefore include those screened positive for an insufficient amount or a deficiency in a functional gene product (protein), or that produce an aberrant, partially functional or non-functional gene product (protein).

Methods of administration or delivery include any mode compatible with a subject. Methods and uses of the invention include delivery and administration systemically, regionally or locally, or by any route, for example, by injection or infusion. Such delivery and administration include parenterally, e.g. intraocularly, intravascularly, intravenously, intramuscularly, intraperitoneally, intradermally, subcutaneously, or transmucosal. Exemplary administration and delivery routes include intravenous (i.v.), intraperitoneal (i.p.), intrarterial, subcutaneous, intra-pleural, intubation, intrapulmonary, intracavity, iontophoretic, intraorgan, intralymphatic. In particular embodiments, an AAV vector is administered or delivered parenterally, such as intravenously, intraarterially, intraocularly, intramuscularly, subcutaneously, or via catheter or intubation.

Doses can vary and depend upon whether the type, onset, progression, severity, frequency, duration, or probability of the disease to which treatment is directed, the clinical endpoint desired, previous or simultaneous treatments, the general health, age, gender, race or immunological competency of the subject and other factors that will be appreciated by the skilled artisan. The dose amount, number, frequency or duration may be proportionally increased or reduced, as indicated by any adverse side effects, complications or other risk factors of the treatment or therapy and the status of the subject. The skilled artisan will appreciate the factors that may influence the dosage and timing required to provide an amount sufficient for providing a therapeutic or prophylactic benefit.

Invention AAV vectors, and other compositions, can be incorporated into pharmaceutical compositions, e.g., a pharmaceutically acceptable carrier or excipient. Such pharmaceutical compositions are useful for, among other things, administration and delivery to a subject in vivo or ex vivo.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Thus, such a pharmaceutical composition may be used, for example in administering a viral vector or viral particle to a subject.

Such compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Compositions suitable for parenteral administration comprise aqueous and non-aqueous solutions, suspensions or emulsions of the active compound, which preparations are typically sterile and can be isotonic with the blood of the intended recipient. Non-limiting illustrative examples include water, saline, dextrose, fructose, ethanol, animal, vegetable or synthetic oils.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

Pharmaceutical compositions and delivery systems appropriate for the compositions, methods and uses of the invention are known in the art (see, e.g., Remington: The Science and Practice of Pharmacy (2003) 20^{th} ed., Mack Publishing Co., Easton, Pa.; Remington's Pharmaceutical Sciences (1990) 18^{th} ed., Mack Publishing Co., Easton, Pa.; The Merck Index (1996) 12^{th} ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11^{th} ed., Lippincott Williams & Wilkins, Baltimore, Md.; and Poznansky et al., Drug Delivery Systems (1980), R. L. Juliano, ed., Oxford, N.Y., pp. 253-315).

A “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. AAV vectors, and pharmaceutical compositions thereof can be packaged in single or multiple unit dosage form for ease of administration and uniformity of dosage.

TABLE 1

| | Target Gene | |
|---|---|---|
| Condition to | for gene | |
| be Treated | therapy | Protein Encoded |
| Retinitis | Rho | Rhodopsin |
| Pigmentosa | PDE6β | Phosphodiesterase 6β |
| | ABCA4 | ATP-binding cassette, sub-family A, |
| | | member 4 |
| | RPE65 | Retinal pigment epithelium-specific |
| | | 65 kDa protein |
| | LRAT | Lecithin Retinal Acyltransferase |
| | RDS/ | Retinal degeneration, slow/Peripherin |
| | Peripherin | |
| | MERTK | Tyrosine-protein kinase Mer |
| | CNGA1 | cGMP-gated cation channel alpha-1 |
| | RPGR | Retinitis pigmentosa GTPase regulator |
| | IMPDH1 | Inosine-5-prime-monophosphate |
| | | dehydrogenase, type I |
| | ChR2 | Channelrhodopsin-2 |
| Maculopathies | GUCY2D | Guanylate Cyclase 2D |
| | RDS/ | Retinal degeneration, slow/Peripherin |
| | Peripherin | |
| | AIPL1 | Aryl-hydrocarbon interacting protein- |
| | | like 1 |
| | ABCA4 | ATP-binding cassette, sub-family A, |
| | | member 4 |
| | RPGRIP1 | Retinitis pigmentosa GTPase regulator |
| | | interacting protein 1 |
| Leber's | IMPDH1 | Inosine-5-prime-monophosphate |
| congenital | | dehydrogenase, type I |
| amaurosis and | AIPL1 | Aryl-hydrocarbon interacting protein- |
| early onset | | like 1 |
| severe retinal | GUCY2D | Guanylate Cyclase 2D |
| dystrophy | LRAT | Lecithin Retinal Acyltransferase |
| | MERTK | Tyrosine-protein kinase Mer |
| | RPGRIP1 | Retinitis pigmentosa GTPase regulator |
| | | interacting protein 1 |
| | RPE65 | Retinal pigment epithelium-specific |
| | | 65 kDa protein |
| | CEP290 | Centrosomal protein of 290 kDa |
| Stargardt | ABCA4 | ATP-binding cassette, sub-family A, |
| disease | | member 4 |
| Usher | DFNB31 | Whirlin |
| Syndrome | MYO7A | Myosin 7A |
| | USH1C | Harmonin |
| | CDH23 | Cadherin 23 |
| | PCDH15 | Protocadherin 15 |
| | USH1G | SANS |
| | CLRN1 | Clarin 1 |
| Achromatopsia | GNAT2 | Guanine nucleotide binding protein, |
| | | alpha transducing activity polypeptide |
| | | 2 |
| | CNGA3 | Cyclic nucleotide gated channel alpha |
| | | 3 |
| | CNGB3 | Cyclic nucleotide gated channel beta |
| | | 3 |
| X-linked | Rs1 | Retinoschisin 1 |
| retinoschisis | | |
| Ocular albinism | OA1 | Ocular albinism type 1 |
| Leber's | MT-ND4 | NADH-ubiquinone oxidoreductase |
| Hereditary | | chain 4 |
| Optic | | |
| Neuropathy | | |
| Oculocutaneous | (OCA1) | Oculocutaneous albinism type 1 |
| albinism | tyrosinase | tyrosinase |
| Glaucoma | p21 WAF-1/ | Cyclin-dependent kinase inhibitor |
| | OCipl | interacting protein 1 |
| Choroideremia | REP-1 | Rab escort protein 1 |
| Age related | PDGF | Platelet-derived growth factor |
| macular | Endostatin | |
| degeneration | Angiostatin | |
| | VEGF | Vascular endothelial growth factor |
| | inhibitor | inhibitor |
| Color blindness | Opsin | Opsin |
| Blue Cone | OPN1LW | Long-wave-sensitive opsin 1 |
| Monochromacy | | |
| Lysosomal | arylsulfatase | Arylsulfatase B |
| storage disease | B | |
| IV | | |
| Lysosomal | β- | β-Glucuronidase |
| storage disease | glucuronidase | |
| VII | | |

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All applications, publications, patents and other references, GenBank citations and ATCC citations cited herein are incorporated by reference in their entirety. In case of conflict, the specification, including definitions, will control.

All of the features disclosed herein may be combined in any combination. Each feature disclosed in the specification may be replaced by an alternative feature serving a same, equivalent, or similar purpose. Thus, unless expressly stated otherwise, disclosed features (e.g., AAV vectors, nucleic acid such as donor nucleic acid, guide RNA, target gene, protein, are an example of a genus of equivalent or similar features.

As used herein, the singular forms “a”, “and,” and “the” include plural referents unless the context clearly indicates otherwise. Thus, for example, reference to “a nucleic acid” includes a plurality of such nucleic acids, and reference to “a vector” includes a plurality of such vectors, such as AAV vectors.

As used herein, all numerical values or numerical ranges include integers within such ranges and fractions of the values or the integers within ranges unless the context clearly indicates otherwise. Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions. Accordingly, the following examples are intended to illustrate but not limit the scope of the invention claimed.

### EXAMPLES

### Example 1

### The Example is a Proposed Strategy for Correction/Replacement of a Target Gene.

To target oversized loci, for example the ABCA4 locus (certain mutations of which result in Stargardt Disease), perform in vitro cleavage of endogenous ABCA4 locus in HEK 293. First, transfect HEK 293 cells with tandem Cas9/gRNA construct targeting intron 16 of ABCA4. Then, detect specific cleavage with Celase I Surveyor Mutagenesis assay (run gel to detect cleavage).

For correction of oversized target genes, for example ABCA4, stably transfect HEK 293 cells with ABCA4 cDNA (or any future target cDNA greater than 4.8 kb using a similar strategy) including intron 16 between exons 16 and 17, upstream of the major mutations (or relevant region of other oversized targets). Intron 16 is targeted with a Flag-tagged cDNA construct with a Splice Acceptor and exons 17-50. Correction is then verified by co-immunoprecipitation and Western blot on ABCA4 (or any future target protein) and Flag protein.

### Example 2

### The Example is a Proposed Strategy for Cleavage of Endogenous Oversized or Disease-Causing Gene Loci.

To target oversized loci, for example the ABCA4 locus (certain mutations of which result in Stargardt Disease), perform in vitro cleavage of endogenous ABCA4 locus in HEK 293 cells in vitro. First, transfect HEK 293 cells in culture with tandem Cas9/gRNA plasmid construct targeting intron16 of ABCA4. Then, detect specific cleavage with Celase I Surveyor Mutagenesis assay, to detect the frequency of cleavage events at that site. This study will show proof of concept for targeting either oversized loci for cleavage and gene insertion or disruption of mutated gene loci that lead to harmful pathology with specifically designed Cas9/gRNA DNA.

In another example, package the above Cas9 and gRNA plasmid(s) into AAV vectors. Transduce HEK 293 cells in culture with AAV construct(s) targeting intron 16 of ABCA4, and detect site-specific cleavage with Celase I Surveyor Mutagenesis assay. This study will show proof of concept for targeting either oversized loci for cleavage or disruption of mutated gene loci with an AAV vector system.

### Example 3

### The Example is a Proposed Strategy for Correction of Endogenous Oversized Loci:

For correction of oversized target genes, for example ABCA4, stably transfect HEK 293 cells with ABCA4 cDNA (or any future target cDNA greater than 4.8 kb using a similar strategy) including intron 16 between exons 16 and 17, upstream of some of the major mutations in Stargardt Disease (or relevant region of other oversized targets). Target intron 16 with a Cas9/gRNA construct for cleavage, and transfect a flag-tagged cDNA construct with a Splice Acceptor and exons 17-50 of ABCA4 for insertion into the cleaved intron. Verify correction by co-immunoprecipitation and Western blot on ABCA4 (or any future target protein) and Flag protein.

### Abbreviations

| | |
|---|---|
| AAV | Adeno-associated Virus |
| Cas | CRISPR-associated protein |
| Cas9 | CRISPR-associated protein 9 |
| cDNA | Complementary DNA |
| CMV | Cytomegalovirus |
| CRISPR | Clustered regularly interspaced short palindromic repeats |
| DNA | Deoxyribonucleic Acid |
| HEK 293 | Human Embryonic Kidney 293 Cells |
| ITR | Inverted Terminal Repeat |
| kb | Kilobase(s) |
| Poly A | Polyadenylation signal |
| RNA | Ribonucleic Acid |
| SA | Splice Acceptor |
| SD | Splice Donor |
| VP1 | Viral Protein 1 |
| VP2 | Viral Protein 2 |
| VP3 | Viral Protein 3 |

### REFERENCES

1. Atchison R W, Casto B C, HAMMON WM. ADENOVIRUS-ASSOCIATED DEFECTIVE VIRUS PARTICLES. Science. 149(3685), 754-756 (1965).
2. Hermonat P L, Muzyczka N. Use of adeno-associated virus as a mammalian DNA cloning vector: transduction of neomycin resistance into mammalian tissue culture cells. Proc Natl Acad Sci USA. 81(20), 6466-6470 (1984).
3. Laughlin C A, Tratschin J D, Coon H, Carter B J. Cloning of infectious adeno-associated virus genomes in bacterial plasmids. Gene. 23(1), 65-73 (1983).
4. Matsushita T, Elliger S, Elliger C, et al. Adeno-associated virus vectors can be efficiently produced without helper virus. Gene Ther. 5(7), 938-945 (1998).
5. Xiao X, Li J, Samulski R J. Production of high-titer recombinant adeno-associated virus vectors in the absence of helper adenovirus. Journal of Virology. 72(3), 2224-2232 (1998).
6. Gonçalves MAFV. Adeno-associated virus: from defective virus to effective vector. ViroL J. 2, 43 (2005).
7. Maguire A M, Simonelli F, Pierce E A, et al. Safety and efficacy of gene transfer for Leber's congenital amaurosis. N. Engl. J. Med. 358(21), 2240-2248 (2008).
8. Maguire A M, High K A, Auricchio A, et al. Age-dependent effects of RPE65 gene therapy for Leber's congenital amaurosis: a phase 1 dose-escalation trial. Lancet. 374(9701), 1597-1605 (2009).
9. Bennett J, Ashtari M, Wellman J, et al. AAV2 gene therapy readministration in three adults with congenital blindness. Science Translational Medicine. 4(120), 120ra15 (2012).
10. Jinek M, Chylinski K, Fonfara I, Hauer M, Doudna J A, Charpentier E. A programmable dual-RNA-guided DNA endonuclease in adaptive bacterial immunity. Science. 337(6096), 816-821 (2012).
11. Qi L S, Larson M H, Gilbert L A, et al. Repurposing CRISPR as an RNA-guided platform for sequence-specific control of gene expression. Cell. 152(5), 1173-1183 (2013).
12. Cong L, Ran F A, Cox D, et al. Multiplex genome engineering using CRISPR/Cas systems. Science. 339(6121), 819-823 (2013).
13. Hsu P D, Lander E S, Zhang F. Development and applications of CRISPR-Cas9 for genome engineering. Cell. 157(6), 1262-1278 (2014).
14. Hsu P D, Scott D A, Weinstein J A, et al. DNA targeting specificity of RNA-guided Cas9 nucleases. Nat Biotechnol. 31(9), 827-832 (2013).
15. Doudna J A, Charpentier E. Genome editing. The new frontier of genome engineering with CRISPR-Cas9. Science. 346(6213), 1258096 (2014).

What is claimed is:

1. A composition comprising:
a) a first AAV vector comprising a nucleic acid encoding a functional Type II CRISPR-Cas9; and/or
b) a second AAV vector comprising a guide RNA sequence specific for a target gene.

2. The composition of claim 1, further comprising c) a third AAV vector comprising a donor nucleic acid sequence for correction or replacement of a target gene.

3. A composition comprising:
a) a first AAV vector comprising a nucleic acid encoding a functional Type II CRISPR-Cas9 and a guide RNA sequence specific for a target gene.

4. The composition of claim 3, further comprising b), a second AAV vector comprising a donor nucleic acid sequence for correction or replacement of a target gene.

5. The composition of claim 1, wherein the first AAV vector further comprises one or more of the following elements, optionally in 5′>3′ orientation:
i) a 5′ AAV inverted terminal repeat (ITR);
ii) a promoter and optional enhancer;
iii) a Cas9 cDNA encoding the functional Type II CRISPR-Cas9;
iv) a polyadenylation signal;
v) a 3′ AAV inverted terminal repeat (ITR).

6. The composition of claim 1 or 2, wherein the second or third AAV vector further comprises one or more of the following elements, optionally in 5′->3′ orientation:
i) a 5′ AAV ITR;
ii) a promoter and optional enhancer;
iii) the guide RNA sequence is specific for a target gene involved in ocular development or function;
iv) a stuffer or filler nucleic acid sequence;
v) a 3′ AAV ITR.

7. The composition of claim 2, wherein the third AAV vector further comprises one or more of the following elements, optionally in 5′->3′ orientation:
i) a 5′ AAV ITR;
ii) a 5′ slice acceptor site;
iii) the donor nucleic acid sequence comprising a sequence for correction or replacement of a gene involved in ocular development or function;
iv) a polyadenylation signal;
v) an AAV 3′ ITR.

8. A method of treating a disease of a subject treatable by disrupting, correcting or replacing a mutated or defective target gene, or a target gene encoding a protein having defective or partial function or activity, comprising administering to the subject a composition of:
a) a first AAV vector comprising a nucleic acid encoding a functional Type II CRISPR-Cas9 and a guide RNA sequence specific for a target gene; and
b) a second AAV vector comprising a donor nucleic acid sequence for correction or replacement of the target gene.

9. A method of treating an ocular disease of a subject treatable by disrupting, correcting or replacing a mutated or defective target gene, or a target gene encoding a protein having defective or partial function or activity, comprising administering to the subject a composition of:
a) a first AAV vector comprising a nucleic acid encoding a functional Type II CRISPR-Cas9 and a guide RNA sequence specific for a target gene; and
b) a second AAV vector comprising a donor nucleic acid sequence for correction or replacement of the target gene.

10. The method of claim 8, wherein the mutated or defective gene, or gene encoding a protein having defective or partial function or activity corrected or replaced is a mutated or defective version of a gene set forth in Table 1, or a version of a gene set forth in Table 1 that encodes a protein having defective or partial function or activity.

11. The method of claim 8, wherein the mutated or defective gene, or gene encoding a protein having defective or partial function or activity disrupted is a mutated or defective version of a gene set forth in Table 1, or a version of a gene set forth in Table 1 that encodes a protein having defective or partial function or activity.

12. The method of claim 8, wherein the mutated or defective gene, or gene encoding a protein having defective or partial function or activity is present in retinal, corneal, scleral or choroid cells.

13. The method of claim 8, wherein the mutated or defective gene, or gene encoding a protein having defective or partial function or activity is replaced.

14. The method of claim 8, wherein the mutated or defective gene, or gene encoding a protein having defective or partial function or activity has one or more mutations corrected or replaced.

15. The method of claim 8, wherein the mutated or defective gene, or gene encoding a protein having defective or partial function or activity is disrupted.

16. The method of claim 8, wherein the mutated or defective gene, or gene encoding a protein having defective or partial function or activity has 1-10 nucleotides or mutations disrupted, corrected or replaced.

17. The method of claim 8, wherein a region of less than about 4.8 kb of the mutated or defective gene, or gene encoding a protein having defective or partial function or activity, is disrupted, corrected or replaced.

18. The method of claim 8, wherein the subject is a mammal.

19. The method of claim 8, wherein the subject is a human.

20. The method of claim 8, wherein the method improves ocular development or function.

21. The method of claim 8, wherein the donor nucleic acid is inserted upstream of a majority of mutations in the target gene.

22. The method of claim 8, wherein the donor nucleic acid is inserted downstream of less frequent mutations in the target gene.

23. The method of claim 8, wherein the donor nucleic acid is inserted into an intron of the target gene.
